(19) United States
(12) Patent Application Publication
(10) Pub. No.: US 2009/0075296 A1
(43) Pub. Date: Mar. 19, 2009

(54) METHODS AND COMPOSITIONS FOR ASSAYING REGULATORY T CELLS

(75) Inventor: David A. Horwitz, Santa Monica, CA (US)

Correspondence Address: MORGAN, LEWIS & BOCKIUS, LLP, ONE MARKET SPEAR STREET TOWER, SAN FRANCISCO, CA, 94105, US

(73) Assignees: University of Southern California, Los Angeles, CA (US); National Institutes of Health, Bethedsa, MD (US)

(21) Appl. No.: 12/194,101

(22) Filed: Aug. 19, 2008

Related U.S. Application Data
(60) Provisional application No. 60/957,046, filed on Aug. 21, 2007.

(51) Int. Cl. G01N 33/53

(52) U.S. Cl. 435/7.2

(57) ABSTRACT

Methods and compositions for determining whether a T cell is a regulatory T cell and for determining whether a population of cells includes at least one regulatory T cell or a cell with the potential to become a regulatory T cell. The invention includes methods and compositions for detecting TGF-β on the surface of a cell. The invention also provides methods and compositions for evaluating the suppressive activity of a regulatory T cell based on the presence and/or the amount of membrane-bound TGF-β.

### CROSS REFERENCE TO RELATED APPLICATIONS

This application claims priority to U.S. Provisional Application Ser. No. 60/957,046, filed on Aug. 21, 2007, the disclosure of which is hereby incorporated by reference in its entirety.

### GOVERNMENTAL SUPPORT OF INVENTION

This invention was made with governmental support under Contract No. R01 A1041768 awarded by the National Institutes of Health. The government has certain rights in the invention.

### TECHNICAL FIELD

The invention is generally related to methods for assessing whether a cell population includes regulatory T cells or cells with the potential to become regulatory T cells. The invention includes assays which detect the presence of membrane-bound TGF-β to assess whether such cells are or have the potential to be regulatory T cells.

### BACKGROUND OF THE INVENTION

Regulatory T cells (also known as suppressor T cells or Treg cells) are a specialized subpopulation of T cells that act to suppress activation of the immune system and thereby maintain immune system homeostasis and tolerance to self-antigens. Regulatory T cells can down-regulate antibody synthesis by lytic or cytokine-mediated mechanisms. The latter involve transforming growth factor-beta (TGF-β) and other inhibitory cytokines (Wahl, S. M. (1994), J Exp Med 180:1587 190).

Treg cells, either naturally occurring or induced by combination of T cells with a regulatory composition, can be used to inhibit graft rejection and to prevent and treat graft versus host disease. Treg cells express several molecules which can be used to characterize a T cell as a Treg cell, including forkhead/winged-helix family transcriptional repressor p3 (Foxp3), the tumor necrosis factor family, and membrane-bound TGF-β (Yi et al., (2006) Cell. & Mol. Immun., 3(3):189-195).

Although there are methods and compositions known to induce regulatory T cell activity and molecules that can be used to characterize a T cell as a Treg cell (also referred to herein as a “Treg”), there are currently no standard methods for detecting or determining whether a T cell is a regulatory T cell. There are also no standard methods for evaluating whether a population of cells includes a regulatory T cell or a cell with the potential to become a regulatory T cell. In order to determine whether a particular protocol is able to effectively induce the formation of Treg cells for use in the treatment and prevention of autoimmune diseases, graft rejection and graft-versus-host disease, a standard assay is needed.

### SUMMARY OF THE INVENTION

Accordingly, the present invention provides methods for determining whether a population of T cells comprises a regulatory T cell or a cell that has the potential to become a regulatory T cell.

In one aspect, the invention provides a method for assessing whether a population of cells includes a regulatory T cell or a potential regulatory T cell. This aspect of the invention includes the step of assaying for TGF-β on a surface on at least one of said population of cells. The presence of TGF-β on the surface of at least one cell of the population indicates that the remainder of the cell population includes at least one regulatory T cell or a potential regulatory T cell.

In another aspect, the invention provides a method for assessing whether a peripheral blood mononuclear cell (PBMC) population includes a regulatory T cell or a potential regulatory T cell. This aspect of the invention includes the steps of (a) isolating a PBMC population; (b)stimulating that PBMC population; (c) separating a portion of the PBMC population from the remainder of the population; and (d) assaying for TGF-β on the surface of at least one of the PBMCs in that portion of the PBMC population. The presence of TGF-β on the surface of at least one PBMC indicates that the remainder of the PBMC population includes at least one regulatory T cell or at least one potential regulatory T cell.

In still another aspect, the invention provides a method for assessing whether a peripheral blood mononuclear cell (PBMC) population includes at least one potential regulatory T cell. This method includes the steps of isolating a PBMC population; stimulating that PBMC population; and separating a portion of the PBMC population from the remainder of said PBMC population. This method further includes the step of maintaining the portion of said PBMC population in an in vitro culture for a predetermined period of time; and assaying for TGF-β on the surface of at least one of the PBMCs in that portion of said PBMC population. The presence of TGF-β on the surface of at least one PBMC indicates that the remainder of the PBMC population includes at least one potential regulatory T cell.

In yet another aspect, the invention provides a method for assessing the ability of donor antigen to induce formation of regulatory T cells or potential regulatory T cells. This method includes the steps of: (a) isolating a peripheral blood mononuclear cell (PBMC) population; (b) contacting the PBMC population with a donor antigen; and (c) assaying for TGF-β on the surface of at least one of the PBMCs in the PBMC population. The presence of TGF-β on the surface of at least one PBMC indicates that the remainder of the PBMC population includes at least one regulatory T cell or at least one potential regulatory T cell. Detecting the presence of TGF-β on the surface of at least one PBMC in this aspect of the invention thereby assesses the ability of the donor antigen to induce formation of a regulatory T cell or potential regulatory T cell.

In still another aspect, the invention provides a method for assessing the ability of a regulatory composition to induce formation of regulatory T cells or potential regulatory T cells. In this aspect, the regulatory T cells or potential regulatory T cells can prevent rejection of a graft. This aspect of the invention includes the steps of isolating peripheral blood mononuclear cells (PBMCs) from a recipient and an organ donor; irradiating T cell-depleted mononuclear cells from the organ donor peripheral blood mononuclear cells; and combining ex vivo the recipient PBMCs and a regulatory composition. In a further aspect, the regulatory composition includes TGF-β and the irradiated organ donor peripheral blood mononuclear cells. This aspect of the invention further includes the step of assaying for TGF-β on the surface of the recipient PBMCs. The presence of TGF-β on the surface of at least one of the PBMCs indicates that the PBMCs include at least one regulatory T cell or at least one potential regulatory T cell that can prevent rejection of a graft.

In another aspect, the invention provides a method for assessing the ability of a regulatory composition to treat donor cells to prevent graft-versus-host disease. In this aspect, a peripheral blood mononuclear cell (PBMC) population is removed from a donor and treated with a regulatory composition for a time sufficient to induce T cell tolerance in the treated PBMC. This method further includes the step of assaying for TGF-β on the surface of at least one of the treated PBMCs, and the presence of TGF-β on the surface of at least one PBMC indicates that the PBMC population includes at least one regulatory T cell or at least one potential regulatory T cell. Detecting the presence of TGF-β on the surface of at least one PBMC thereby assesses the ability of the regulatory composition to treat donor cells to prevent graft-versus-host disease.

### BRIEF DESCRIPTION OF THE DRAWINGS

FIG. 1 demonstrates that activated natural CD4+CD25+FoxP3+ cells can co-express membrane bound TGF-β.

FIG. 2 demonstrates that activated CD4+CD25+ Tregs express a TGF-β LAP complex.

FIG. 3A and FIG. 3B demonstrate that both natural and induced Tregs produce (A) membrane-bound and (B) soluble TGF-β.

FIG. 4 demonstrates that human naive CD4+ cells express a complex of membrane-bound TGF-β and Latency Associated Protein after repeated stimulation for three weeks.

FIG. 5 demonstrates temperature dependence of antibody staining for membrane-bound TGF-β.

FIG. 6 demonstrates the frequency of expression of CD4+CD25+ and Foxp3+ in mice with lupus across a period of time

FIG. 7. demonstrates that CD4+CD25+ cells from mice with lupus express lower amounts of membrane-bound TGF-β as they grow older.

### DETAILED DESCRIPTION OF THE INVENTION

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### Overview

The present invention provides methods and compositions for determining whether a population of cells comprises a regulatory T cell (“Treg”) or a cell that has the potential to be a regulatory T cell (“potential Treg”). In one aspect, the invention provides assays for detecting TGF-β on the surface of a cell, wherein the presence of TGF-β on the surface of a cell indicates that the cell is a Treg or a potential Treg. In a further aspect, such assays include the use of antibodies directed to a TGF-β protein. In a still further aspect, the assays of the invention are conducted on populations of peripheral blood mononuclear cells (“PBMCs”). In one embodiment, these PBMCs are treated with a regulatory composition before they are assayed for the presence of Tregs and/or potential Tregs.

### Cell Populations Comprising Regulatory T Cells

Assays of the invention can be used with any population that could comprise Tregs and/or potential Tregs. Potential Tregs are cells that may not yet have full suppressive capability, but nevertheless express markers or possess other characteristics that indicate that such cells could become Tregs upon further stimulation.

In one aspect, assays of the invention are applied to peripheral blood mononuclear cells (“PBMCs”). In a further aspect, these PBMCs are isolated from a subject. Methods of isolating such cells from a subject are well known in the art and also described in U.S. Pat. Nos., 6,228,359; 6,358,506; 6,759,035; 6,797,267; 6,447,765; 6,803,036; and 7,115,259 as well as co-pending U.S. application Ser. Nos. 10/194,344; 10/772,768; 11,507,908; 11/241,467; 11/400,950; and 11/394,761, all of which are hereby incorporated in their entirety for all purposes, and in particular for their teachings regarding isolating PBMCs from a subject.

In an exemplary embodiment, assays of the invention are applied to populations of T cells that have been enriched for one or more types of T cells, including but not limited to CD4⁺, CD8⁺, CD8⁻CD4⁻, and NK T cells.

In one exemplary embodiment, the subject from which PBMCs or other cell populations of use with the present invention are isolated is a mammal. In a further exemplary embodiment, the subject is a human.

In another aspect, assays of the invention are used with PBMCs that have been activated. In an exemplary embodiment, these activated PBMCs include naturally occurring Tregs. In another exemplary embodiment, these activated PBMCs include induced Tregs. A number of methods for inducing the formation of regulatory T cells exist, as described in, for example, U.S. Pat. Nos., 6,228,359; 6,358,506; 6,759,035; 6,797,267; 6,447,765; 6,803,036; and 7,115,259 as well as co-pending U.S. application Ser. Nos. 10/194,344; 10/772,768; 11,507,908; 11/241,467; 11/400,950; and 11/394,761, all of which are hereby incorporated in their entirety for all purposes, and in particular for their teachings regarding the formation of regulatory T cells.

In one aspect, assays of the invention are used to detect the presence of Tregs and/or potential Tregs in a population of PBMCs that have been treated with a regulatory composition. By “regulatory composition” herein is meant a composition that can cause the formation of regulatory T cells when cultured with PBMCs with or without an antigen. Generally, these compositions comprise TGF-β alone or in combination with a cytokine (including without limitation IL-2, IL-4, IL-10, IL-15 and/or TNIα), T cell activators, and/or antigens from a donor or from a recipient. Regulatory compositions that can be used to form regulatory T cells are known in the art and described for example in U.S. Pat. Nos., 6,228,359; 6,358,506; 6,759,035; 6,797,267; 6,447,765; 6,803,036; and 7,115,259 as well as co-pending U.S. application Ser. Nos. 10/194,344; 10/772,768; 11,507,908; 11/241,467; 11/400,950; and 11/394,761, all of which are hereby incorporated in their entirety for all purposes, and in particular for their teachings regarding such regulatory compositions.

By “transforming growth factor -β” or “TGF-β” herein is meant any one of the family of the TGF-βs, including the three isoforms TGF-β1, TGF-β2, and TGF-β3; see Massague, J. (1990), J. Ann. Rev. Cell Biol 6:597. Lymphocytes and monocytes produce the β1 isoform of this cytokine (Kehrl, J. H. et al. (1991), Int J Cell Cloning 9: 438-450). The TFG-β can be any form of TFG-β that is active on the mammalian cells being treated. TGF-β can be purchased from Genzyme Pharmaceuticals, Farmington, Mass.

By “treated” herein is meant that the cells are incubated with the regulatory composition for a time period sufficient to develop the capacity to inhibit immune responses, including antibody and autoantibody production, particularly when the cells are transferred into a subject. The incubation will generally be under physiological temperature.

In one embodiment, a population of cells is assayed by testing an aliquot of the population for the presence of TGF-β on the surface of one or more cells of the aliquot. Detection of TGF-β on the surface of one or more cells of the aliquot indicates that the remainder of the population of cells includes at least one Treg or potential Treg. In a further embodiment, the population of cells comprises resting T cells that have not been stimulated to induce Tregs. In this embodiment, the aliquot of cells is activated using methods known in the art and described herein, and the activated aliquot is then subjected to assays of the invention to detect the presence of TGF-β on the surface of one or more cells of the aliquot. This embodiment of the invention provides the ability to determine if a particular method of inducing Tregs is successful on the cells contained in the aliquot. If multiple protocols for inducing Tregs are to be tested using the assays of the invention, testing aliquots rather than the full population avoids the necessity of having to isolate cells from the subject multiple times.

### Assays for Regulatory T Cells

As discussed herein, assays of the invention provide the ability to test the whether certain methods, including methods in which PBMCs are treated with a regulatory composition, are successful in forming Tregs or potential Tregs. Assays of the invention may also be used as a diagnostic tool, particularly for diseases related to Treg production, including for example immune and autoimmune diseases.

In one aspect, assays of the invention include the step of determining whether a cell expresses TGF-β on its surface (also referred to herein as “membrane-bound TGF-β”). It has been shown that cells comprising membrane-bound TGF-β display suppressive activity. (see Oida et al., (2003) J. Immunol., 170(5): 2516-2522; Oida et al., (2006) J. Immunol., 177(4):2331-2339; and Nakamura et al., (2001), J. Experimental Med., 194(5): 629-44, each of which is hereby incorporated in its entirety for all purposes, and in particular for discussion of cells expressing membrane-bound TGF-β). Thus, a cell that expresses TGF-β on its surface can be identified using assays of the invention as a Treg or a potential Treg.

Assays of the invention can be conducted on any population of cells that could comprise Tregs or potential Tregs, including PBMCs and splenocytes. Although the following discussion of assays of the invention may be directed primarily to PBMCs for the sake of clarity, it will be understood that the assays of the invention are not limited to use solely with PBMCs.

In one aspect, the invention provides methods for assessing whether a population of cells comprises a Treg or a potential Treg by assaying for TGF-β on the surface of at least one of the population of cells. Detection of TGF-β on the surface of one of such a population of cells indicates that the cell population includes at least one Treg or potential Treg.

Detection and quantification of membrane-bound proteins are well known in the art. Many means are known in the art for detecting the presence of proteins on the surface of one or more cells and are within the scope of the present invention.

In one aspect, assays of the invention utilize antibodies directed to TGF-β. Such antibodies are well known in the art. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In one aspect, assays of the invention analyze antibody staining of cells using flow cytometry. Such flow cytometry methods are well known and well-characterized in the art.

TGF-β antibodies of use with the present invention may be purchased from commercial sources or may be generated using art-recognized methods. It will be appreciated that any antibody directed to TGF-β will be useful in assays of the invention. In an exemplary aspect, antibodies used with assays of the invention recognize epitopes of the extracellular portion of membrane-bound TGF-β. In another exemplary aspect, the TGF-β antibodies are monoclonal antibodies.

In one aspect, assays of the invention include the steps of applying a TGF-β antibody to a population of cells and detecting whether the antibody is able to stain the cells. Cells stained by the antibody express TGF-β on their surface. Detection of such stained cells can be accomplished using methods known in the art. In one exemplary embodiment, cells stained for surface TGF-β are detected using flow cytometry. In a further embodiment, the cells are also stained for other proteins, including without limitation, latency-associated peptide, CD-3, CD28, FoxP3. In a still further embodiment, the cells are stained for any markers of Tregs. It will be appreciated that stains for both membrane-bound and intracellular proteins can be utilized in accordance with the present invention.

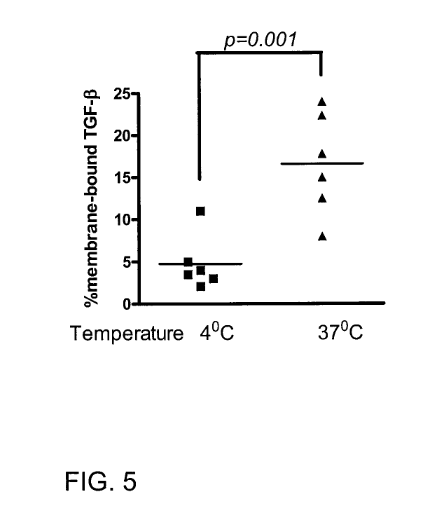

In another aspect, assays of the invention include methods for staining for surface TGF-β with TGF-β antibodies. Such staining methods are known in the art. In one aspect, the antibody staining procedure is conducted at 37° C., which results in a more sensitive assay for TGF-β than is seen at standard staining temperatures. The improved sensitivity of the present assays conducted at 37° C. as compared to the standard 4° C. is shown in FIG. 5, which illustrates that the 37° C. assay detected a greater percentage of cells with membrane-bound TGF-β. Standard antibody staining practices are not generally conducted at temperatures higher than 4° C., because many markers show decreased expression at higher temperatures, which can limit the sensitivity and specificity of such assays.

In a further aspect of the invention, TGF-β antibody staining assays are conducted at temperatures in the range of about 0° C. to about 45° C., of about 4° C. to about 40° C., of about 8° C. to about 35° C., of about 12° C. to about 30° C., and of about 16° C. to about 25° C. It will be appreciated that the temperature at which the staining is conducted can be optimized to increase the sensitivity of the assay and ensure that most if not all cells in a population expressing membrane-bound TGF-β are detected.

In a still further aspect of the invention, propidium iodide is applied to identify dead cells in the population that could otherwise non-specifically label with the TGF-β antibody. Addition of the propidium iodide can improve the specificity of the staining procedures used in accordance with any of the assays of the invention as described herein.

In a further aspect of the invention, staining with TGF-β antibodies is conducted in combination with staining with one or more other antibodies. Such antibodies include without limitation antibodies directed to CD4, CD25, CD28, forkhead/winged-helix family transcriptional repressor p3 (Foxp3), tumor necrosis factor, as well as any other markers of resting T cells or activated Tregs. Staining with multiple antibodies can be conducted simultaneously or sequentially in any order. As discussed above regarding antibodies for TGF-β, staining with other antibodies can be conducted across a range of temperatures, and can be optimized using methods known in the art and described herein to improve the specificity of the assay for the proteins of interest.

In a further aspect, assays of the invention detect both membrane-bound and soluble TGF-β.

In one aspect, assays of the invention are conducted on an aliquot of a population of cells. In such an assay, detection of membrane-bound TGF-β on at least one cell of the aliquot indicates that the remainder of the population of cells includes at least on Treg or potential Treg.

In an exemplary embodiment, the population of cells includes PBMCs isolated from a subject. In a further embodiment, the PBMCs are activated with a regulatory composition. In a still further embodiment, the PBMCs are isolated from a subject that is a mammal.

In one aspect, the invention provides a method for assessing whether a PBMC population includes a Treg or a potential Treg. In this aspect, the method includes the steps of isolating a PBMC population from a subject, stimulating that PBMC population, and then assaying for TGF-β on the surface of at least on PBMC. The method may also include the step of separating an aliquot of the PBMC population from the remainder of the population either before or after the stimulating step. If the aliquot is separated before the stimulating step, the stimulating and later assessment steps can be conducted on the aliquot itself, or on both the aliquot and the remainder of the PBMC population either simultaneously or sequentially in any order. In this aspect, the PBMC population and/or the aliquot is assayed for TGF-β on the surface of at least one PBMC. Detection of membrane-bound TGF-β on at least one PBMC indicates that the remainder of the PBMC population comprises at least one Treg or potential Treg.

In an exemplary embodiment, cell populations to be assayed may be stimulated by application of a regulatory composition. In a further embodiment, the regulatory composition comprises TGF-β. In a still further embodiment, the regulatory composition can also include other components as described in the art and herein.

In one aspect, prior to any assaying for membrane-bound TGF-β, a PBMC population is maintained in an in vitro culture for a predetermined period of time. This predetermined period of time can range from about 5 hours to about 6 weeks. In one embodiment, the cells are maintained in culture for at least four weeks prior to being assayed for membrane-bound TGF-β. In a further embodiment, the cells are maintained in culture from about 1 day to about 5 weeks, from about 5 days to about 4 weeks, from about 1 week to about 3 weeks, and from about 10 days to about 2 weeks.

In a further aspect, only an aliquot of the PBMC population is maintained in culture for a predetermined period of time and then assayed for membrane-bound TGF-β. In this exemplary aspect, the remainder of the PBMC population can be stored, until it is determined from assays conducted on the aliquot that the remainder of the PBMC population is likely to contain at least one Treg or potential Treg. The stored PBMCs can then be utilized in any downstream applications as are known in the art, including prevention and treatment of undesirable or aberrant immune responses, prevention and treatment of graft-versus-host disease, and prevention and treatment of graft rejection. Such downstream uses are further described in U.S. Pat. Nos., 6,228,359; 6,358,506; 6,759,035; 6,797,267; 6,447,765; 6,803,036; and 7,115,259 as well as co-pending U.S. application Ser. Nos. 10/194,344; 10/772,768; 11,507,908; 11/241,467; 11/400,950; and 11/394,761, all of which are hereby incorporated in their entirety for all purposes, and in particular for their teachings regarding uses of Tregs.

In a further aspect, the PBMC population (or aliquot of the PBMC population) that is maintained in culture for a predetermined period of time is re-stimulated at least once during that pre-determined period of time. In one exemplary embodiment, the cells are re-stimulated with a T cell activator and/or a cytokine. In a further exemplary embodiment, the T cell activator is anti-CD3, anti-CD28, or a mitogen. In another exemplary embodiment, the cytokine is IL-2. In a still further embodiment, the re-stimulation is accomplished without further application of TGF-β. In another embodiment, the re-stimulation includes another application of TGF-β.

In one embodiment, the PBMC population or aliquot that is maintained in culture is re-stimulated between 1 and 10 times during the predetermined period of time that the cells are maintained in culture. In another exemplary embodiment, the cells are re-stimulated between 2 and 8, between 3 and 6, and between 4 and 5 times during the predetermined period of time that the cells are maintained in culture.

In one aspect, the invention provides a method for assessing the ability of a donor antigen to induce formation of a Treg or potential Treg. A donor antigen can be any antigen derived from a donor that (1) induces the formation of a recipient's regulatory T cells or (2) boosts the recipient Treg population when administered to the recipient. In this aspect, PBMCs treated with a donor antigen are assayed for TGF-β on their surface, and the presence of TGF-β on the surface of at least one PBMC indicates that the remainder of the PBMC population includes at least one Treg or potential Treg. In this aspect of the invention, the presence of cells comprising membrane-bound TGF-β after treatment with a donor antigen indicates that the donor antigen can induce the formation of a Treg or a potential Treg.

In another aspect, the assays of the invention can be used to assess the ability of a regulatory composition to induce a Treg or potential Treg, wherein the Treg or potential Treg can prevent rejection of a graft. In this aspect, PBMCs may be isolated from a recipient and an organ donor. T-cell depleted mononuclear cells from the donor are irradiated and combined ex vivo with a regulatory composition and the recipient PBMCs. In an exemplary embodiment, the regulatory composition includes TGF-β. Assays of the invention can then be conducted to determine if the recipient PBMCs express TGF-β on their surface. The presence of TGF-β on the surface of at least one recipient PBMC indicates that the regulatory composition is able to induce formation of a Treg or potential Treg that can prevent rejection of a graft.

In still another aspect, the assays of the invention can be used to assess the ability of a regulatory composition to treat donor cells for preventing graft-versus-host disease. In such an aspect, the donor cells may be a PBMC population removed from a donor which is then treated with a regulatory composition for a time sufficient to induce T cell tolerance in the cells of the PBMC population. The treated PBMCs can then be assayed for membrane-bound TGF-β. The presence of TGF-β on the surface of at least one of the treated PBMCs indicates that the treated population includes at least one Treg or potential Treg, and therefore further provides an assessment of the ability of the regulatory composition to treat donor cells to prevent graft-versus-host disease.

Although the present invention is described in some detail for purposes of illustration, it will be readily appreciated that a number of variations known or appreciated by those of skill in the art may be practiced within the scope of present invention. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes.

### EXAMPLES

### Example 1

### Activated Natural CD4+CD25+FoxP3+ Cells can Co-Express Membrane-TGF-β

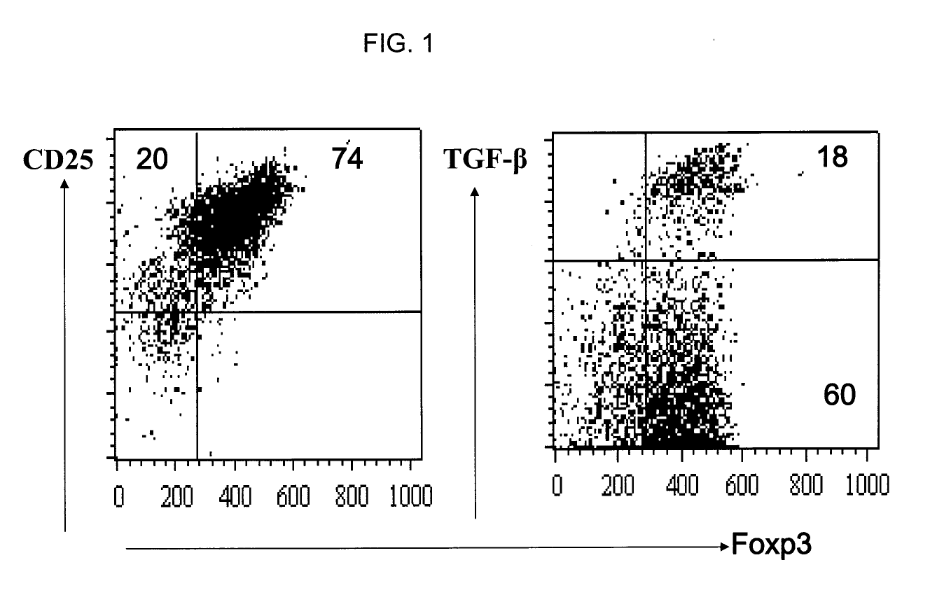

CD4+CD25+ cells were isolated from mouse cells (C57BL/6 spleen cells) using methods known in the art and described herein. Purified CD4+CD25+ cells were stimulated with anti-CD3/28 beads in the absence of TGF-β—1 bead per 5 cells and IL-2 (20 u/ml)—for three days. These cells were stained for CD25, and were also stained for surface TGF-β and intracellular Foxp3. FIG. 1 shows that a subset of the FoxP3+ cells will stain for membrane TGF-β. According to the results in FIG. 1, it would be expected that the cells represented by the data in the upper right quadrant of the right-hand plot have the greatest suppressor activity, as these cells stain for Foxp3 and membrane-TGF-β. Comparing the left-hand and right-hand plot indicates that the assay for membrane-TGF-β is a more sensitive and specific assay for detecting suppressor cells (Tregs) than simply staining for CD25, as not all the cells positive for both CD25 and Foxp3 express membrane-TGF-β. As discussed herein, it has been shown that suppressor cells express membrane-TGF-β.

Detection of membrane TGF-β in this assay was dependent on the temperature at which the cells were stained. Although it is general practice to stain cells at 4° C., for the assays of the present invention, optimal TGF-β staining was seen at 37° C. (see Example 5 and FIG. 5). Results in FIG. 1 are representative of three independent experiments.

### Example 2

### Activated Tregs Express a TGF-β/Latency Associated Protein Complex

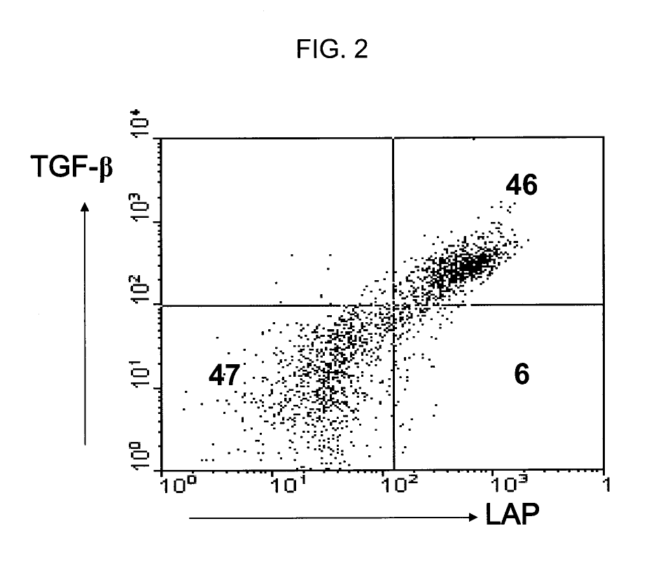

CD4+CD25+ cells were also isolated and stimulated as described above in Example 1. The membrane-bound TGF-β and LAP were stained and detected by Flow Cytometry. Results of such an experiment are shown in FIG. 2, which shows that TGF-β is bound to Latency Associated Protein. This experiment was repeated with twice with similar results. Cells expressing LAP have been shown to have greater suppressive activity than those that do not express this protein. (se Oida et al., (2003) J. Immunol., 170(5)). FIG. 2 thus shows that assaying for membrane-TGF-β provides a sensitive method for identifying cells with increased suppressive activity.

### Example 3

### nTreg and iTreg can Display Membrane Bound TGF-β and can Produce Soluble Active TGF-β

Natural CD4+CD25+ regulatory T cells (nTregs) were prepared by isolating CD4+CD25+ cells. Induced CD4+CD25+ cells (iTregs) were prepared by isolating CD4+CD25− cells and collecting the cells that stained positive for L selectin (CD62L).

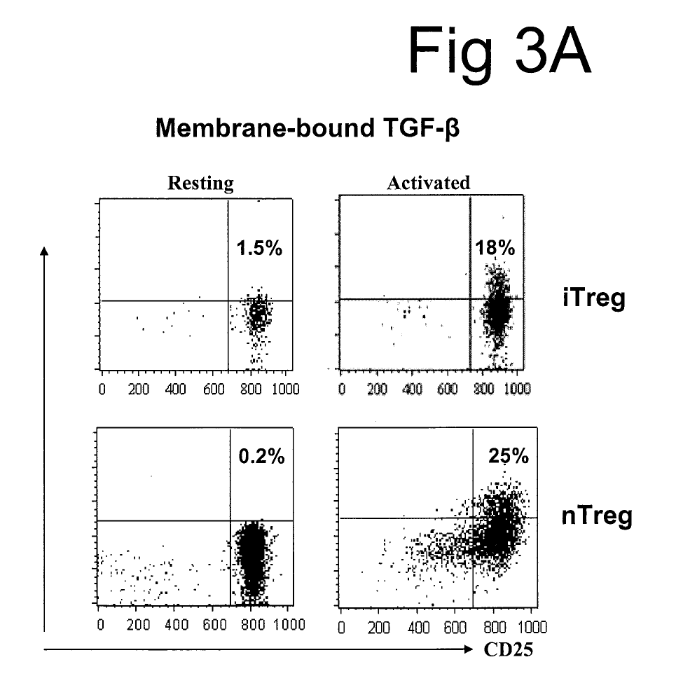

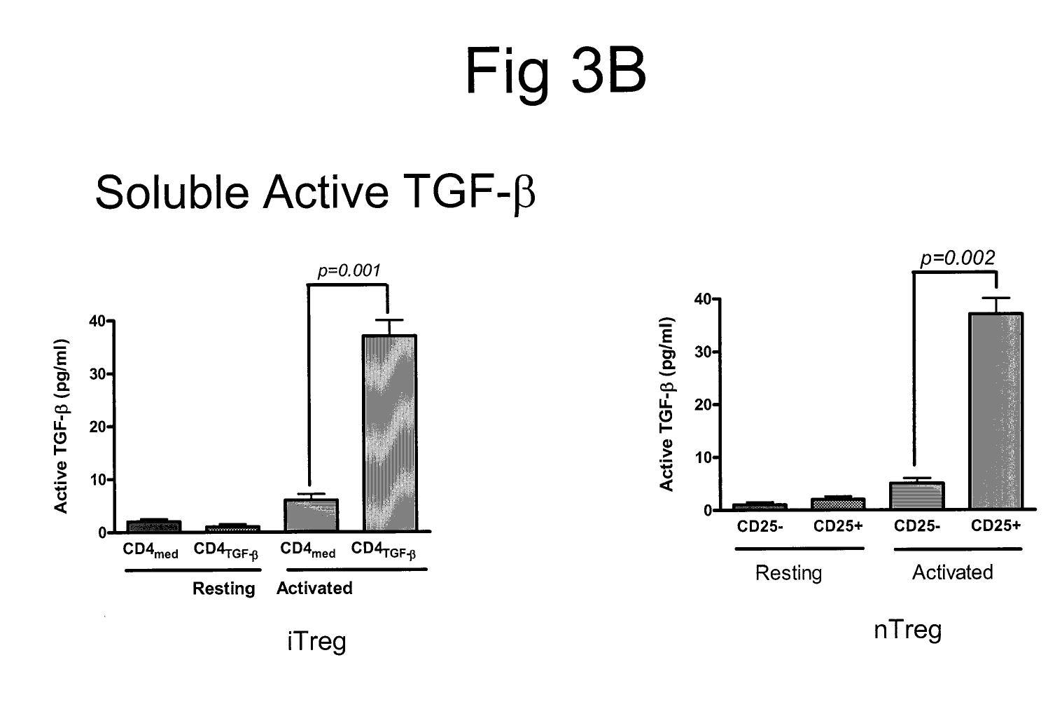

Naïve CD4+CD25− cells were stimulated with anti-CD3/28 beads, IL-2 (20 U/ml) and TGF-β1 (2 ng/ml) for 5 days to induce them to become CD25+Foxp3+ regulatory T cells (iTregs). Both nTregs and iTregs were stimulated with anti-CD3/28 beads in the presence of IL-2 (20 U/ml) for 3 days in serum free AIMV medium. The cells were extensively washed. A portion of the cells were stained for membrane TGF-β. A second portion of the cells were re-stimulated with anti-CD3/28 beads for 3 to 5 days and then assayed for active TGF-β in the culture medium. Membrane-bound TGF-β was determined by FACS analysis and soluble active TGF-β from the supernatants was determined by mink lung epithelial cells transfected with a luciferase gene construct. Several concentrations of TGF-β were included to generate a standard curve, and the variation between triplicate samples was always <10%. FIG. 3 shows that both nTregs and iTregs show increased membrane-bound and soluble TGF-β upon activation. The result shown is one of five independent experiments.

### Example 4

### Human Naïve CD4+ Cells Express Membrane TGF-β after Repeated TCR Stimulation for Three Weeks

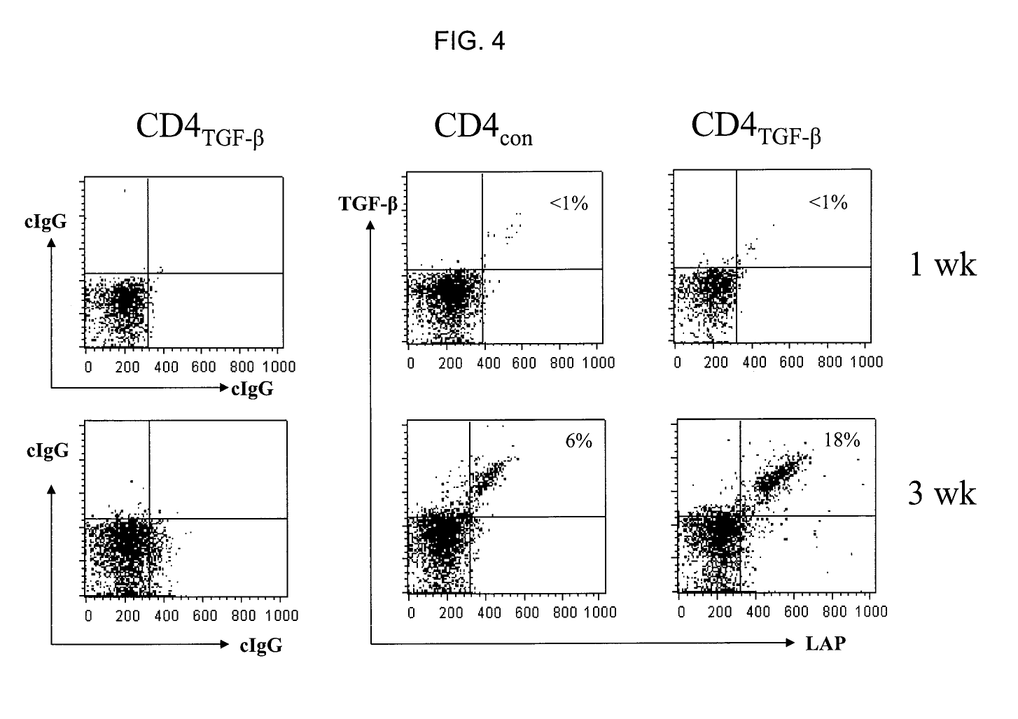

Human naïve CD4+ cells were negatively selected from PBMC and stimulated with anti-CD3/CD28 coated beads (1:20) with IL-2 and TGF-β for five days. The beads were removed and these cells were collected and recultured with IL-2 alone (20 u/ml) for two days (iTregs). The cells were restimulated with anti-CD3/CD28 coated beads (1:10) and IL-2 (20 u/ml) without TGF-β for five days and recultured with IL-2 alone for 2 days. The procedure was repeated and cells collected at 3 weeks. Cells on the first week and third weeks were analyzed by flow cytometry for the co-expression of membrane-bound TGF-β and LAP (see FIG. 4). Priming CD4+ cells with TGF-β during the first week of culture markedly increased the percentage of cells expressing membrane-bound TGF-β later (compare the top and bottom plots of the right-most column in FIG. 4). Results are representative of three independent experiments.

### Example 5

### Effect of Temperature on Membrane-Bound TGF-β Expression by CD4+ Regulatory T Cells

Tregs were induced using the method described above in Example 4. The Tregs were stained with anti-CD4 and anti-CD25 antibodies for 30 minutes at 4° C. and then were stained with anti-TGF-β antibody at either 4° C. or 37° C. for three hours (see FIG. 5). The staining procedure included propidium iodide to gate out dead cells. TGF-β surface expression on CD25+ on the CD4+ gate was determined using flow cytometry. The results from 6 different donors are shown in FIG. 5.

### Example 6

### Decreased Expression of Membrane-Bound TGF-β in Mice with Lupus

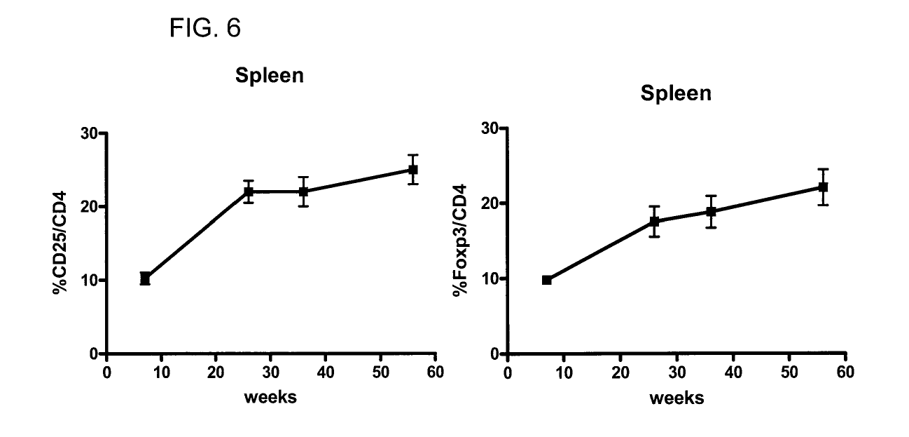

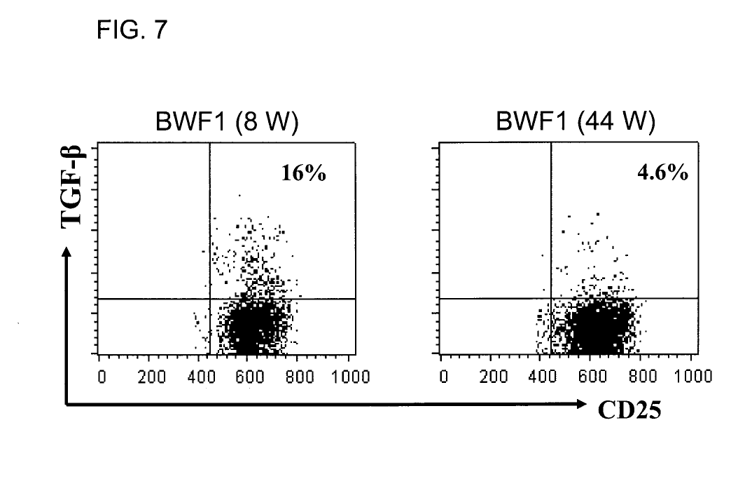

(New Zealand White×New Zealand Black) F1 mice have been shown to spontaneously develop a lupus-like disease that strongly resembles human systemic lupus erythematosus (SLE). Although the percentage of splenic CD4+CD25+ cells and CD4+ cells that express Foxp3 actually increase in these mice with again (FIG. 6), the percentage of CD4+CD25+ cells that express membrane-bound TGF-β decreases almost four-fold as they grow older and develop lupus. (FIG. 7—compare the left-hand plot at 8 weeks with the right-hand plot at 44 weeks). The data in FIG. 7 were obtained from cells stimulated as described in the above Examples and indicate that membrane-bound TGF-β is a better marker for the development of SLE than expression of CD25 or Foxp3 on CD4+ cells, because the changes in expression of membrane-bound TGF-β are better correlated with the onset and progression of the disease than other markers generally used in the art.

What is claimed is:

1. A method for assessing whether a population of cells comprises a regulatory T cell or a potential regulatory T cell, said method comprising assaying for TGF-β on a surface on at least one of said population of cells, wherein the presence of TGF-β on the surface on said at least one of said population of cells indicates that the remainder of said cell population comprises at least one regulatory T cell or a potential regulatory T cell.

2. The method of claim 1, wherein said assaying is accomplished on an aliquot of said population of cells.

3. The method of claim 1, wherein said population of cells comprises peripheral blood mononuclear cells isolated from a subject and activated with a regulatory composition.

4. The method of claim 3, wherein said subject is a mammal.

5. The method of claim 1, wherein said assaying comprises applying a TGF-β antibody to said population of cells.

6. The method of claim 5, wherein said assaying is accomplished at 37° C.

7. A method for assessing whether a peripheral blood mononuclear cell (PBMC) population comprises a regulatory T cell or a potential regulatory T cell, said method comprising:
a. isolating a PBMC population;
b. stimulating said PBMC population;
c. separating a portion of said PBMC population from the remainder of said PBMC population; and
d. assaying for TGF-β on the surface of at least one of the PBMCs in said portion of said PBMC population, wherein the presence of TGF-β on the surface of at least one PBMC indicates that the remainder of said PBMC population comprises at least one regulatory T cell or at least one potential regulatory T cell.

8. The method of claim 7, wherein said stimulating comprises applying a regulatory composition.

9. The method of claim 8, wherein said regulatory composition comprises TGF-β.

10. The method of claim 7, wherein prior to step (d), said portion of said PBMC population is maintained in an in vitro culture for a predetermined period of time.

11. The method of claim 10, wherein said PBMC population comprises human PBMCs.

12. The method of claim 10, wherein said predetermined period of time is at least four weeks.

13. The method of claim 10, wherein during said predetermined period of time, said portion of said PBMC population is re-stimulated at least once with a T cell activator, with a cytokine, or with both a T cell activator and a cytokine.

14. The method of claim 7, wherein said assaying comprises applying a TGF-β antibody to said portion of said PBMC population.

15. The method of claim 14, wherein said assaying is accomplished at 37° C.

16. A method for assessing whether a peripheral blood mononuclear cell (PBMC) population contains at least one potential regulatory T cell, said method comprising:
a. isolating a PBMC population;
b. stimulating said PBMC population;
c. separating a portion of said PBMC population from the remainder of said PBMC population;
d. maintaining said portion of said PBMC population in an in vitro culture for a predetermined period of time; and
e. assaying for TGF-β on the surface of at least one of the PBMCs in said portion of said PBMC population, wherein the presence of TGF-β on the surface of at least one PBMC indicates that the remainder of said PBMC population comprises at least one potential regulatory T cell.

17. The method of claim 16, wherein said isolating step comprises isolating said PBMC population from a human patient.

18. The method of claim 16, wherein said predetermined period of time is one week.

19. The method of claim 16, wherein said PBMCs are human PBMCs and said predetermined period of time is about four weeks.

20. The method of claim 16, wherein during said predetermined period of time, said PBMCs are re-stimulated at least once with a T cell activator, a cytokine, or both a T cell activator and a cytokine.

21. The method of claim 15, wherein said assaying comprises applying a TGF-β antibody to said portion of said PBMC population.

22. The method of claim 21, wherein said assaying is accomplished at 37° C.

23. A method for assessing the ability of donor antigen to induce formation of regulatory T cells or potential regulatory T cells, said method comprising:
a. isolating a peripheral blood mononuclear cell (PBMC) population;
b. contacting said PBMC population with a donor antigen; and
c. assaying for TGF-β on the surface of at least one of the PBMCs in said PBMC population, wherein the presence of TGF-β on the surface of at least one PBMC indicates that the remainder of said PBMC population comprises at least one regulatory T cell or at least one potential regulatory T cell, thereby assessing ability of said donor antigen to induce formation of a regulatory T cell or potential regulatory T cell.

24. A method for assessing ability of a regulatory composition to induce formation of regulatory T cells or potential regulatory T cells, wherein said regulatory T cells or said potential regulatory T cells can prevent rejection of a graft, said method comprising
a. isolating peripheral blood mononuclear cells (PBMCs) from a recipient and an organ donor;
b. irradiating T cell-depleted mononuclear cells from said organ donor peripheral blood mononuclear cells;
c. combining ex vivo said recipient PBMCs and a regulatory composition comprising TGF-β and said irradiated organ donor peripheral blood mononuclear cells; and
d. assaying TGF-β on surface of said recipient PBMCs, wherein said presence of TGF-β on surface of at least one of said PBMCs indicates that said PBMCs comprise at least one regulatory T cell or at least one potential regulatory T cell that can prevent rejection of a graft.

25. A method for assessing ability of a regulatory composition to treat donor cells to prevent graft-versus-host disease, said method comprising:
a. removing a peripheral blood mononuclear cell (PBMC) population from a donor;
b. treating said PBMC population with a regulatory composition for a time sufficient to induce T cell tolerance in said treated PBMC; and
c. assaying for TGF-β on surface of at least one of the treated PBMCs, wherein the presence of TGF-β on the surface of at least one PBMC indicates that said PBMC population comprises at least one regulatory T cell or at least one potential regulatory T cell,
thereby assessing ability of said regulatory composition to treat donor cells to prevent graft-versus-host disease.
